(19) United States
(12) Patent Application Publication
(10) Pub. No.: US 2026/0259144 A1
(43) Pub. Date: Sep. 3, 2026

(54) Visualized Surface Plasmon Resonance Biological Detection Apparatus

(71) Applicant: Ming Chi University of Technology, New Taipei City (TW)

(72) Inventors: Li-Chen Su, Taipei City (TW); Yu-Ching Huang, New Taipei City (TW); Shun-Wei Liu, Taoyuan City (TW)

(21) Appl. No.: 19/215,872

(22) Filed: May 22, 2025

(30) Foreign Application Priority Data
Mar. 3, 2025 (TW) 114107601

(51) Int. Cl. G01N 21/64; G01N 33/543

(52) U.S. Cl. CPC G01N 21/648; G01N 33/54373

(57) ABSTRACT

A visualized surface plasmon resonance biological detection apparatus defines an optical path and includes a first optical module, a surface plasmon resonance biosensor, and a second optical module. The first optical module is disposed on the optical path and is used to provide an infrared light beam. The surface plasmon resonance biosensor is disposed on the optical path, is used to reflect the infrared light beam to generate a reflected light beam, and includes an optical prism and a sensing member that includes a metal layer and an active layer which is spaced apart from the optical prism. The metal layer is connected to the active layer and is disposed between the active layer and the optical prism. The second optical module is disposed on the optical path to convert the reflected light beam into visible light and includes an up-converter.

### CROSS-REFERENCE TO RELATED APPLICATION

This application claims priority to Taiwanese Patent Application No. 114107601, filed Mar. 3, 2025, the disclosure of which is hereby incorporated by reference in its entirety.

### BACKGROUND OF THE DISCLOSURE

### Field of the Disclosure

The disclosure relates to a biological detection apparatus, and more particularly to a visualized surface plasmon resonance biological detection apparatus.

### Description of Related Art

A surface plasmon resonance biosensor is a biomedical detection device that combines surface plasmon resonance technique with a biochip. Because the surface plasmon resonance biosensor is highly sensitive and label-free of fluorescent dye, detection time is greatly reduced, and therefore the surface plasmon resonance biosensor is widely used in the biomedical field.

A detection method for biological samples is disclosed in the journal, Sensors (2022, 22, 2901). The detection method adapts a surface plasmon resonance biosensor to detect a biological sample, and in cooperation with an optical detector, a signal processor electrically connected to the optical detector, and a display electrically connected to the signal processor, a detection result may be visualized on the display. Specifically, the detection method for biological samples includes steps of introducing the biological sample into the surface plasmon resonance biosensor, and allowing the biological sample to interact with a reactant on a metal layer of the surface plasmon resonance biosensor. Then, the surface plasmon resonance biosensor is irradiated with visible light at an angle of incidence, so that the metal layer of the surface plasmon resonance biosensor is excited by the visible light, thereby triggering a surface plasmon resonance phenomenon, and reflected light is generated. Then, the reflected light is received by the optical detector which includes a charge-coupled apparatus (CCD), and is converted into an electrical signal by the optical detector. Next, the electrical signal is processed by the signal processor, and a wavelength and intensity of the reflected light are displayed on the display.

Although by virtue of the optical detector, the signal processor, and the display, the detection result of the surface plasmon resonance biosensor may be visualized, such device is not easy to assemble and carry, and has a high cost. Moreover, because the device is relatively large in size, the device may not be compatible with all spaces, so on-site measurements may not be easy to conduct. In addition, the device converts optical signals into electrical signals, and then converts the electrical signals into readable data in terms of light wavelength and light intensity. Presentation of data in such way involves relatively more steps. Furthermore, although the reflected light is visible to the naked eye, dark pattern regions generated by the surface plasmon resonance phenomenon are relatively large. Therefore, when used to detect different concentrations of biological samples, the dark pattern regions corresponding to the biological samples may be overlapped, which is not conducive to clearly identifying changes and differences in the concentrations of the biological samples with the naked eye.

### SUMMARY OF THE DISCLOSURE

Therefore, an object of the disclosure is to provide a visualized surface plasmon resonance biological detection apparatus that can alleviate at least one of the drawbacks of the prior art.

According to the disclosure, the visualized surface plasmon resonance biological detection apparatus defines an optical path and includes a first optical module, a surface plasmon resonance biosensor, and a second optical module.

The first optical module is disposed on the optical path and is used to provide an infrared light beam.

The surface plasmon resonance biosensor is disposed on the optical path, is adapted for reflecting the infrared light beam to generate a reflected light beam, and includes an optical prism and a sensing member that includes a metal layer and an active layer which is spaced apart from the optical prism. The metal layer is connected to the active layer and is disposed between the active layer and the optical prism.

The second optical module is disposed on the optical path to convert the reflected light beam into visible light and includes an up-converter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other features and advantages of the disclosure will become apparent in the following detailed description of the embodiment(s) with reference to the accompanying drawings. It is noted that various features may not be drawn to scale.

FIG. 1 is a perspective view of an embodiment of a visualized surface plasmon resonance biological detection apparatus according to the disclosure.

FIG. 2 is a schematic view of the embodiment.

FIG. 3 is a schematic view of a surface plasmon resonance biosensor of the embodiment.

FIG. 4 is a photograph illustrating a plurality of dark stripe regions corresponding to different concentrations of biological samples when the embodiment is used to perform detection.

FIG. 5 is a photograph illustrating a plurality of dark stripe regions corresponding to different concentrations of biological samples when red light is used to perform detection.

### DESCRIPTION OF NON-LIMITING EMBODIMENTS OF THE DISCLOSURE

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

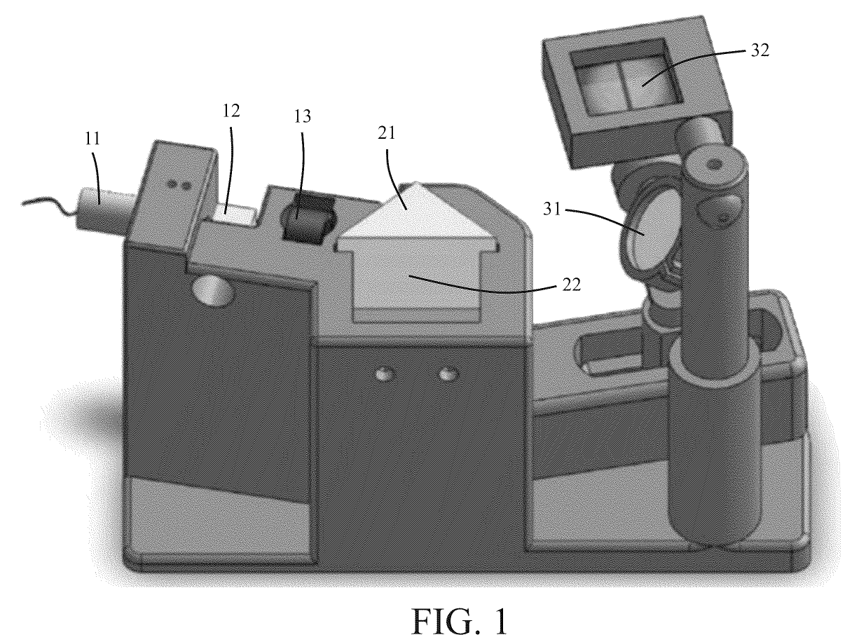

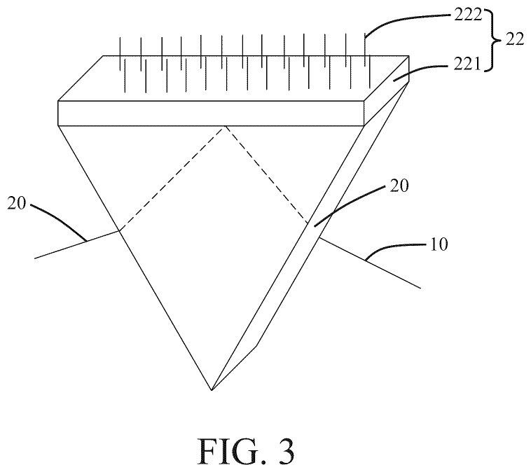

Referring to FIG. 1 to FIG. 3, an embodiment of a visualized surface plasmon resonance biological detection apparatus according to the present disclosure is adapted for detecting a biological sample that includes an analyte. The analyte may be, for example but not limited to, a protein, etc. The analyte may be, for example but not limited to, small molecule ribonucleic acids related to the African swine fever virus, e.g., ssc-mir-122 and ssc-mir-199a-5p.

The visualized surface plasmon resonance biological detection apparatus defines an optical path, and includes a first optical module 1, a surface plasma resonance biosensor 2, and a second optical module 3.

The first optical module 1 is disposed on the optical path, and includes a light source supplier 11, a polarizing beam splitter 12, and a light converging lens 13.

The light source supplier 11 is adapted for emitting infrared light 101. The infrared light 101 may be, for example but not limited to, near-infrared light. A wavelength of the near-infrared light is, for example but not limited to, 940 nm or 980 nm. The light source supplier 11 may be, but not limited to, a light emitter including an infrared light-emitting diode or a light emitter including an infrared light laser diode. The infrared light laser diode is, for example, an infrared laser diode manufactured by Thorlabs™ that emits infrared light having a wavelength of 980 nm.

The polarizing beam splitter 12 is adapted for polarizing the infrared light 101 from the light source supplier 11 so as to obtain a P polarized light 102. The polarizing beam splitter 12 includes a polarizer. The polarizer is, for example but not limited to, a polarizer having an extinction ratio of 1000:1.

The light converging lens 13 is adapted for forming the P polarized light 102 from the polarizing beam splitter 12 into an infrared light beam 10. The light converging lens 13 may be, for example but not limited to, a light converging lens having a focal length of 30 mm.

The surface plasmon resonance biosensor 2 is disposed on the optical path, and is adapted for emitting a reflected light beam 20 when the surface plasmon resonance biosensor 2 that interacts with the analyte of the biological sample is irradiated with the infrared light beam 10 at a predetermined angle of incidence. The surface plasmon resonance biosensor 2 includes an optical prism 21 and a sensing member 22. In some embodiments, the optical prism 21 and the sensing member 22 are spaced apart from each other, i.e., in an Otto configuration. In some embodiments, the optical prism 21 is connected to the sensing member 22, i.e., in a Kretschmann configuration.

The optical prism 21 is, for example but not limited to, a triangular prism. The sensing member 22 includes an active layer 222 for interacting with the analyte of the biological sample, and a metal layer 221 disposed between the optical prism 21 and the active layer 222. The active layer 222 includes a binding agent that may interact with the biological sample, and the binding agent is for specifically binding to the biological sample. The binding agent is, for example but not limited to, a binding agent including biotin and a bioprobe covalently bound to the biotin. The biotin is, for example, streptavidin. The bioprobe is, for example, deoxyribonucleic acid. The metal layer 221 is, for example but not limited to, a gold layer. The sensing member 22 is, for example but not limited to, a streptavidin immobilized sensor chip.

The second optical module 3 is disposed on the optical path, and includes a reflector 31 and an up-converter 32.

The reflector 31 is adapted for changing a direction of the reflected light beam 20 from the surface plasmon resonance biosensor 2 so as to increase an optical path length of the reflected light beam 20. The reflector 31 may be, for example but not limited to, a flat mirror, a Plano-convex lens, or other suitable optical elements.

The up-converter 32 is adapted for converting the reflected light beam 20 from the reflector 31 into visible light 30. The up-converter 32 may be a conventional up-converter, such as, but not limited to, an organic up-converter. For example, the organic up-converter includes a first electrode, a hole blocking layer, a charge generation layer, a hole transport layer, a light emitting layer, an electron transport layer, and a second electrode that are sequentially stacked in such order. The visible light 30 may be green light.

The first electrode is, for example but not limited to, an indium tin oxide (ITO) electrode.

The hole blocking layer may be, for example but not limited to, a hole blocking layer including zinc oxide (ZnO) and polyethylenimine ethoxylated (PEIE).

The carrier-generating layer may be, for example but not limited to, a carrier-generating layer including poly(2,6′-4,8-di(5-ethylhexylthienyl)benzo[1,2-b; 3,3-b]dithiophene) (abbreviated as PTB7-Th) and 2,2′-((2Z,2′Z)-(((4,4-bis(2-ethylhexyl)-4H-cyclopenta[2,1-b: 3,4-b′]dithiophene-2,6-diyl)bis(4-(2- ethylhexyloxy)thiophene-5,2-diyl))bis(methanylylidene))bis(5,6-difluoro-3-oxo-2,3-dihydro-1H-indene-2,1- diylidene))dimalononitrile (abbreviated as COTIC-4F).

The hole transport layer may be, for example but not limited to, a hole transport layer including 9,9′-diphenyl-9H,9′H-3,3′-bicarbazole (abbreviated as BCzPh, CAS. No. 57102-62-2).

The light emitting layer may be, for example but not limited to, a light emitting layer including BCzPh, 3′,3′″,3′″-(1,3,5-triazine-2,4,6-triyl)tris([1,1′-biphenyl]3-carbonitrile) (abbreviated as CN-T2T, CAS. No. 1872292-95-9) and bis(2-phenylpyridine)(acetylacetonate)iridium(III) (abbreviated as Ir(ppy)₂(acac), CAS No. 337526-85-9).

The electron transport layer may be, for example but not limited to, an electron transport layer including CN-T2T.

The second electrode may be, for example but not limited to, a composite electrode including a lithium fluoride (LiF) layer and an aluminum layer.

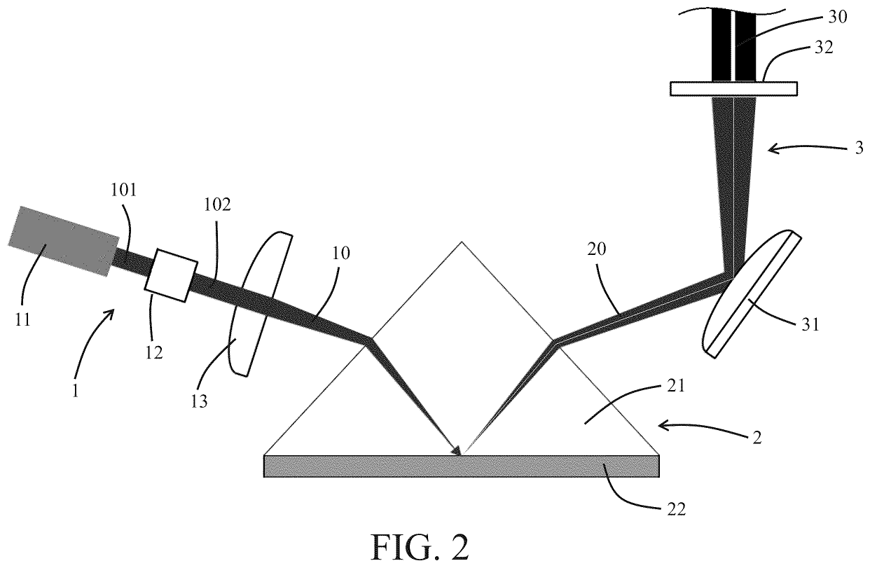

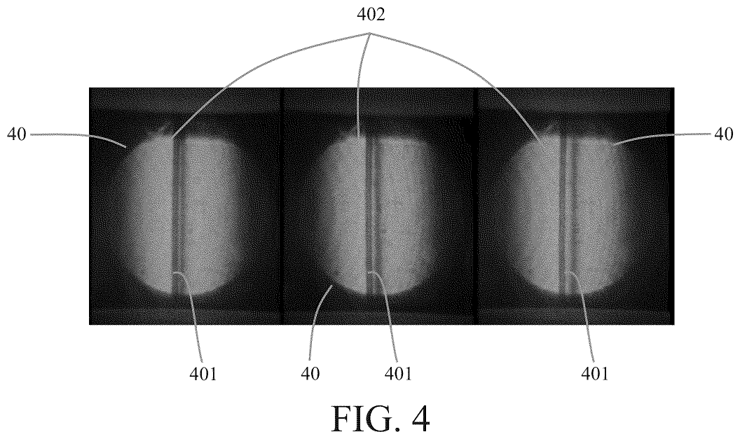

Referring to FIGS. 2 to 4, operation of the visualized surface plasmon resonance biological detection apparatus is described as follows. The light source supplier 11 emits the infrared light 101, and the infrared light 101 travels toward the polarizing beam splitter 12. The polarizing beam splitter 12 receives the infrared light 101 and polarizes the infrared light 101 to generate the P polarized light 102. The P polarized light 102 travels to the light converging lens 13 and is converted into the infrared light beam 10 by the light converging lens 13. The infrared light beam 10 travels to the surface plasmon resonance biosensor 2, and enters the optical prism 21 of the surface plasmon resonance biosensor 2 at a specific angle of incidence. At the same time, the biological sample is introduced to interact with the sensing member 22 of the surface plasmon resonance biosensor 2. When the surface plasmon resonance biosensor 2 that interacts with the analyte of the biological sample is excited by the infrared light beam 10, a surface plasmon resonance phenomenon occurs. At the same time, the surface plasmon resonance biosensor 2 emits the reflected light beam 20. The reflected light beam 20 travels to the reflector 31, is reflected by the reflector 31, and travels to the up-converter 32. The up-converter 32 absorbs the reflected light beam 20 and converts the reflected light beam 20 into the visible light 30. Due to the surface plasmon resonance phenomenon, specific infrared light of the infrared light beam 10 is absorbed, so that the reflected light beam 20 does not include a reflected beam originated from the specific infrared light. Therefore, when the reflected light beam 20 is converted into the visible light 30 by the up-converter 32, a dark stripe region 401 called a surface plasmon resonance dip appears in a visible light display region 40, as shown in FIG. 4. As a concentration of the analyte in the biological sample increases, the surface plasmon resonance phenomenon correspondingly changes, thereby causing the dark stripe region 401 to shift. The greater the concentration is, the greater the degree of the shift, as shown in FIG. 4 (from left to right). To clearly see the degree of the shift of the dark stripe region 401, a user may mark a reference region 402 in the visible light display region 40 first. The reference region 402 is based on a dark stripe region (not shown in FIG. 4) generated when a biological sample without an analyte is being analyzed.

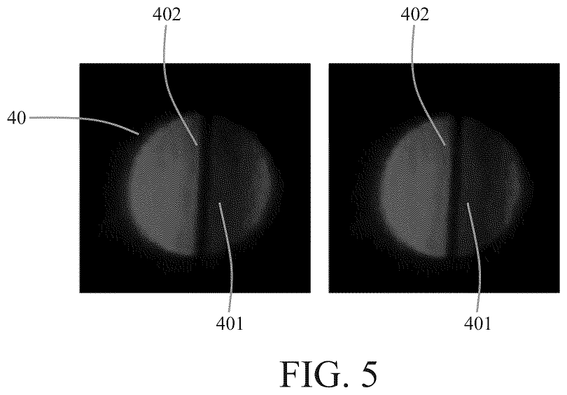

Furthermore, to demonstrate that a detection result including differences in the concentrations of the biological samples obtained by the visualized surface plasmon resonance biological detection apparatus may be seen with the naked eye, the light source supplier 11 of the visualized surface plasmon resonance biological detection apparatus of the present disclosure is replaced with a red light source supplier, the up-converter 32 is omitted, and red light having a wavelength of 635 nm is used. The detection result is shown in FIG. 5. Referring to FIG. 4, the dark stripe regions 401 generated by the visualized surface plasmon resonance biological detection apparatus of the present disclosure corresponding to the different concentrations of the biological samples are narrower and clear, and the shift of the dark stripe regions 401 is easy to see. On the other hand, referring to FIG. 5, concentration of the biological sample on the left is lower than concentration of the biological sample on the right. The dark stripe regions 401 corresponding to both of the biological samples on the left and the right are wide and unclear, so it is difficult to see the differences in the concentrations of the biological samples. Therefore, the visualized surface plasmon resonance biological detection apparatus of the present disclosure can indeed detect the different concentrations of the biological samples and allow the detection results to be seen with the naked eye.

In summary, by virtue of the first optical module 1 which provides the infrared light 101 and the up-converter 32 of the second optical module 3, the visualized surface plasmon resonance biological detection apparatus of the present disclosure may visualize the detection results without equipment such as the optical detector, the signal processor, and the display. The detection results are visualized and may be seen with the naked eye. At the same time, the visualized surface plasmon resonance biological detection apparatus of the present disclosure may perform on-site measurements directly and simplify data collection, thereby reducing dependence on complex computing apparatus. In addition, the visualized surface plasmon resonance biological detection apparatus of the present disclosure may generate narrower dark stripe regions. Therefore, when used to detect the different concentrations of the biological samples, the dark stripe regions corresponding to the biological samples do not overlap, thereby allowing clear identification of changes and the differences in the concentrations of the biological samples by the naked eye.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A visualized surface plasmon resonance biological detection apparatus defining an optical path and comprising:
a first optical module disposed on the optical path and used to provide an infrared light beam; and
a surface plasmon resonance biosensor disposed on the optical path, adapted for reflecting the infrared light beam to generate a reflected light beam, and including
an optical prism, and
a sensing member that includes a metal layer and an active layer which is spaced apart from said optical prism, said metal layer being connected to said active layer and disposed between said active layer and said optical prism; and
a second optical module disposed on the optical path to convert the reflected light beam into visible light, and including an up-converter.

2. The visualized surface plasmon resonance biological detection apparatus as claimed in claim 1, wherein said up-converter is an organic up-converter.

3. The visualized surface plasmon resonance biological detection apparatus as claimed in claim 2, wherein said organic up-converter includes a first electrode, a hole blocking layer, a charge generation layer, a hole transport layer, a light emitting layer, an electron transport layer, and a second electrode that are sequentially stacked in such order.

4. The visualized surface plasmon resonance biological detection apparatus as claimed in claim 1, wherein said optical prism is spaced apart from said sensing member.

5. The visualized surface plasmon resonance biological detection apparatus as claimed in claim 1, wherein said optical prism is connected to said sensing member.

6. The visualized surface plasmon resonance biological detection apparatus as claimed in claim 1, wherein said optical prism is a triangular prism.

7. The visualized surface plasmon resonance biological detection apparatus as claimed in claim 1, wherein said metal layer is a gold layer.

8. The visualized surface plasmon resonance biological detection apparatus as claimed in claim 1, wherein said first optical module includes a light source supplier, a polarizing beam splitter, and a light converging lens.

9. The visualized surface plasmon resonance biological detection apparatus as claimed in claim 1, wherein said second optical module further includes a reflector that is used to increase an optical path length of the reflected light beam from said surface plasmon resonance biosensor.
